(19) United States
(12) Patent Application Publication
(10) Pub. No.: US 2013/0109898 A1
(43) Pub. Date: May 2, 2013

(54) PROCESS FOR REPROCESSING SPENT STYRENE CATALYSTS

(71) Applicant: BASF SE, Ludwigshafen (DE)

(72) Inventors: Florina Corina Patcas, Ludwigshafen (DE); Yong Liu, Ludwigshafen (DE); Martin Dieterle, Ludwigshafen (DE)

(73) Assignee: BASF SE, Ludwigshafen (DE)

(21) Appl. No.: 13/664,525

(22) Filed: Oct. 31, 2012

Related U.S. Application Data
(60) Provisional application No. 61/553,264, filed on Oct. 31, 2011.

(51) Int. Cl. B01J 23/94; B01J 35/02; C07C 5/333; B01J 38/68

(52) U.S. Cl. 585/445; 502/24; 502/100; 585/444

(57) ABSTRACT

The invention relates to a process for producing a new styrene catalyst from a spent styrene catalyst.

The invention relates to a process for producing a new styrene catalyst from a spent styrene catalyst.

In view of significantly increased prices for rare earth compounds, there is a great interest in the selective recovery of rare earth compounds from spent catalysts. For the recovery of cerium compounds, one option is spent iron oxide-containing catalysts, as used for dehydrogenation of hydrocarbons, for example for dehydrogenation of isopentene to isopentadiene (isoprene) or of ethylbenzene to styrene. These generally have a cerium dioxide content in the range from 1 to 25% by weight, especially of 5 to 15% by weight. Such catalysts are described, for example, in EP 1 027 028 A1, DE 101 54 718 A1 and EP 0 894 528 B1.

A challenge in the processing of such spent catalysts is the composition thereof, which is complex in some cases. For instance, the catalysts comprise, as well as compounds of the rare earth metals, usually also compounds of numerous further elements, such as iron, potassium, molybdenum, magnesium, calcium, tungsten, titanium, copper, chromium, cobalt, nickel, vanadium and others.

For example, styrene catalysts consist principally of oxides of the elements iron and potassium, the iron being present predominantly as the trivalent oxide. In addition, styrene catalysts include various elements such as cerium, molybdenum, tungsten, vanadium, calcium, magnesium, etc. in oxidic form as promoters. The cerium is present in the styrene catalyst in the form of cerium dioxide CeO₂ in considerable amounts of about 1 to 25% by weight. During the dehydrogenation of ethylbenzene to styrene, the trivalent iron (Fe(III)) is partly reduced to divalent iron (Fe(II)). This forms magnetite Fe₃O₄. During utilization, styrene catalysts gradually lose activity. Several processes are responsible for the deactivation, for example potassium loss through vaporization, the irreversible formation and deposition of carbon or the increasing reduction of trivalent iron to divalent iron. After about 2 to 4 years, the deactivated styrene catalysts are deinstalled from the reactors and replaced by new catalysts. As a result, large amounts of spent deinstalled styrene catalysts arise globally every year, the reprocessing of which is of great economic interest due to the constantly rising costs of the doping components.

The prior art gives several descriptions of the reutilization of deinstalled styrene catalysts. In EP 1919614, WO 94/11104, CN 101306375, CN 101623643 and DD 268631, the deinstalled catalyst is optionally ground, calcined, possibly mixed with fresh raw materials (iron oxides, potassium compounds, promoters), extruded to shaped bodies and calcined again. This involves subjecting the deinstalled catalyst present therein to one to two additional calcining steps, usually at quite high temperatures of up to 1000° C. As a consequence, there is a reduction in the specific surface area of the catalyst compared to a catalyst which is produced exclusively from fresh raw materials. As known to those skilled in the art, a lower specific surface area in a catalyst leads to a smaller number of accessible active sites and consequently to a lower activity. The styrene catalysts produced by the route described from deinstalled catalysts therefore often have the disadvantage that their activity is lower compared to catalysts which have been produced exclusively from fresh raw materials.

WO 2007/009927 A1 describes a process for producing dehydrogenation catalysts using secondary raw material which is obtained by processing spent catalysts. In this process, there is no chemical separation of the catalyst constituents into compounds of the individual metals. Instead, 10 to 70% by weight of a calcined and ground spent catalyst comprising iron oxide are mixed with 30 to 90% by weight of a catalyst material comprising fresh iron oxide.

KR2002-0093455 describes a process for obtaining cerium dioxide from spent dehydrogenation catalysts which are used for preparation of styrene from ethylbenzene. This involves moist grinding of the spent catalyst, for example in a ball mill, down to a particle size of 0.1 to 5 μm. After removal of the ground catalyst by filtration and addition of acid to the solids, a slurry is prepared with a pH of 0.5 to 3.0, which is filtered once again. The solid residue is admixed with water and, with addition of organic dispersants, dispersed using ultrasound, whereupon two phases form, the intention being that the lower phase comprises iron oxide (magnetite) and the upper phase cerium dioxide. The very fine grinding of the spent catalyst is costly and inconvenient; the organic dispersants used can pollute the wastewater.

It is an object of the present invention to provide a process for producing a new styrene catalyst from a spent styrene catalyst, wherein the specific surface area of the new styrene catalyst shall be at least the same as that of the spent catalyst.

This object is achieved by a process for reprocessing spent styrene catalysts, in which a spent catalyst is brought completely into solution and its constituents are isolated in solid form from the solution and used to produce a new styrene catalyst.

The invention thus provides a process for producing a new styrene catalyst from a spent styrene catalyst, comprising the steps of
(i) treating the spent styrene catalyst with an aqueous acid and optionally a reducing agent to obtain an aqueous solution comprising the soluble constituents of the catalyst or a suspension,
(ii) converting the aqueous solution or suspension to a solid,
(iii) adding any missing constituents after analysis,
(iv) shaping and calcining the solid mixture obtained in step (iii).

In general, spent cerium-containing catalysts which are reprocessed by the process according to the invention comprise compounds at least of the following metals:

iron, corresponding to 40 to 90% by weight of Fe₂O₃,

potassium, corresponding to 1 to 40% by weight of K₂O, and

cerium, corresponding to 1 to 25% by weight of CeO₂, especially 5 to 15% by weight of CeO₂.

In a preferred embodiment, the catalysts additionally comprise compounds of

calcium, corresponding to 0 to 10% by weight of CaO,

molybdenum, corresponding to 0.1 to 10% by weight of MoO₃, and/or

magnesium, corresponding to 0 to 10% by weight of MgO.

In addition, the catalysts may comprise small amounts of further elements present as typical impurities in the iron oxides used as primary starting materials.

In one embodiment of the process according to the invention, the catalysts comprise oxides of iron, potassium, and cerium. In a further embodiment, the catalysts comprise oxides of iron, potassium, molybdenum, cerium and calcium. In a further embodiment, the catalysts comprise oxides of iron, potassium, molybdenum, cerium, calcium and magnesium.

Prior to the treatment of the spent styrene catalyst with an aqueous acid in the first step (ii) of the process according to the invention, the styrene catalyst can be heated (calcined) in an oxygenous atmosphere, in order to oxidatively remove any carbon and organic residues present. The oxygenous gas used is preferably air, but it is also possible to use lean air. This calcination step can take place at temperatures between 400° C. and 1100° C., preferably between 500 and 1000° C., more preferably between 600 and 900° C. The catalyst can be calcined in a stationary manner on metal sheets in a muffle furnace, or in a rotary tube oven or in a fluidized bed. In general, the calcination will also oxidize the magnetite present in the catalyst to hematite, i.e. all Fe(II) cations are converted to Fe(III) cations. In addition, any Ce(III) compounds present are also converted to CeO₂.

In addition, the spent styrene catalyst prior to the treatment with an aqueous acid in the first step (i) of the process according to the invention can optionally be washed with water at pH values of 7 to 12. This removes the potassium compounds from the catalyst, as a result of which the acid for the later neutralization of the potassium is saved and the typical variation in the potassium content and the amount of bound anions (for example chloride, sulfate, nitrate) in the spent catalyst is reduced.

Likewise prior to the treatment with an aqueous acid, the spent catalyst can be mechanically comminuted, for instance by grinding or crushing. A lower particle size is favorable for the subsequent dissolution stage. Of course, the mechanical comminution of the catalyst may also follow the heating. In a preferred embodiment of the invention, the spent catalyst is comminuted in such a way that the mean particle diameter is in the range from 1 to 700 μm, preferably 5 to 500 μm, especially from 10 to 200 μm. The sequence of the comminution, washing and heating steps is as desired. Preference is given to first comminuting the spent catalyst, then washing and finally heating it.

In the first step (i) of the process according to the invention, the spent styrene catalyst is treated with an aqueous acid, for example an inorganic acid (e.g. hydrochloric, sulfuric, nitric acid) or organic acid (e.g. acetic acid, formic acid, ascorbic acid, citric acid etc.) or a mixture of two or more acids, at pH values of <0.5. This at least partly converts the metal oxides present in the catalyst to salts, and they go into solution. Whether the catalyst is entirely or partly dissolved can be decided via the ratio of acid to catalyst (stoichiometric/substoichiometric) and via the reaction time. If the intention is to fully dissolve the deinstalled catalyst, a slightly superstoichiometric amount (10 to 20% acid excess) is used, and the reaction is conducted until all of the solids have dissolved. If only partial dissolution is intended, a substoichiometric or just stoichiometric amount (0 to 50% acid deficiency) is used and the reaction time is kept sufficiently short. The partial dissolution of the spent catalyst, more particularly of the iron oxide, may be sufficient to increase the specific surface area of the catalyst without consuming an unnecessary amount of chemicals.

In general, a spent styrene catalyst will comprise sufficient iron(II) compounds to reduce the insoluble cerium dioxide CeO₂ present to soluble cerium(III) compounds. It may also be necessary in some cases, however, to use a reducing agent, such as hydrogen peroxide, formic acid, oxalic acid, hydrazine or other reducing agents customary in industry, in order to convert the cerium dioxide fully to the soluble Ce(III) salt of the respective acid.

The concentration of the aqueous acid may, for example, be from 5 to 99.9% by weight, preferably from 10 to 80% by weight, more preferably from 25 to 75% by weight. The pH of the aqueous acid is −2 to 0.5, preferably from −1.5 to 0.4, more preferably from −1.1 to 0. The pH of the acidic solution can rise with increasing dissolution of constituents of the spent catalyst.

The stoichiometric ratio of acid to catalyst may be from 10 to 200%, preferably between 20 and 150%, more preferably between 50 and 120%. The stoichiometric ratio is understood to mean the theoretical ratio of acid to the metal ions to be dissolved in the catalyst, which is required for formation of stable metal salts at the treatment temperature.

To accelerate the dissolution, the acid solution can be heated under reflux at a temperature of 10° C. up to 120° C. Preference is given to a temperature between 20° C. and 80° C., more preferably between 20° C. and 60° C. The dissolution process is performed within a period between 0.5 h and 24 h. This process step produces an aqueous solution or a suspension in which some or all metal cations from the spent catalyst are in the form of dissolved salts.

In the course of dissolution, any carbonates present in the spent catalyst form carbon dioxide. In order to control foam formation, the dissolution can be performed in several ways. For example, the deinstalled catalyst can be initially charged in demineralized water and concentrated acid can be added stepwise until the desired acid:catalyst ratio is attained. Alternatively, the full amount of acid can be initially charged in demineralized water and the catalyst can be added in portions.

The weight ratio between the deinstalled catalyst solids and the amount of acidic liquid is kept between 1:1 and 1:20, in order to enable good dispersion and mixing of the solids. Preference is given to a ratio between 1:2 and 1:15, more preferably between 1:5 and 1:10.

In the second step (ii) of the process according to the invention, the solution or suspension obtained by the acid treatment of the deinstalled catalyst is converted to a solid, for example by spray drying or precipitation and optional oxidizing calcination. The previously dissolved metal cations are present in this solid, for instance, as oxides, oxide hydroxides, hydroxides or carbonates.

In one embodiment of the process according to the invention, the acidic solution or suspension is precipitated up to a pH of 5 to 12 by addition of a hydroxide, carbonate or hydrogencarbonate of an alkali metal (e.g. Li, Na, K) or alkaline earth metal (e.g. Ca, Mg) or of ammonia. Preference is given to establishing a final pH between 7 and 10. The precipitation product is separated from the liquid phase (filtration, centrifugation) and washed repeatedly with demineralized water. In another variant of the process, the water is withdrawn from the acidic solution or suspension. For this purpose, for example, the water can be vaporized or the solution can be spray dried. The solids formed can be washed once or more than once with demineralized water, in order to remove any inorganic anions present, for example chloride, sulfate or nitrate. Thereafter, the solid can be calcined under air at a temperature of 300° C. to 1000° C. This decomposes and/or oxidizes the metal hydroxides or other metal salts present to form the respective metal oxides.

The solid obtained in the second step (ii) of the process according to the invention can serve as a precursor for a new styrene catalyst. For this purpose, in the third step (iii) of the process according to the invention, after analysis, any missing constituents and promoters, for example potassium in the form of oxides, hydroxides, carbonates or other compounds, can be added in order to obtain the desired catalyst composition.

In a preferred embodiment of the invention, the new catalyst comprises compounds of the following elements (contents based on the element oxides): iron −50 to 90% by weight, potassium −1 to 30% by weight, cerium −1 to 20% by weight, molybdenum −0 to 10% by weight, tungsten −0 to 10% by weight, vanadium −0 to 10% by weight, magnesium −0 to 10% by weight, calcium −0 to 10% by weight and 0 to 10% by weight of oxides of other metals such as Cr, Co, Ni, Cu, Zn, Ag, Pt, Pd, Al, La or other promoters known in the literature, where the contents add up to 100% by weight. In addition, it is possible to add assistants to the catalyst precursor, in order to improve the processibility, the mechanical strength and the pore morphology of the catalyst. For example, it is possible to add potato starch, graphite, cellulose, alginates, stearic acid, and also portland cement, kaolin, montmorillonite or waterglass.

In the fourth step (iv) of the process according to the invention, the solids mixture obtained in step (iii) is processed by standard methods for shaping (kneading, extruding, drying) and calcining to give a new catalyst. For example, the solids mixture can be tableted, shaped in a pelletizing drum to pellets, or mixed with water or an aqueous solution of sugar, starch, polyvinyl alcohol, polyvinylpyrrolidone or the like in a mixer or kneader, and then extruded to various shapes. Examples of shaped bodies are cylinders, rings, stars and honeycombs.

After the shaping, the possibly moist shaped bodies are dried at temperatures of 50° C. to 500° C. Preference is given to using temperatures of 80 to 350° C. The drying may take place, for example, in drying cabinets on metal sheets, in drying drums or on belt driers. The subsequent calcination of the catalyst is performed preferably in a rotary tube furnace at temperatures between 500° C. and 1100° C., preferably between 700 and 1000° C.

The new catalyst obtained by the process according to the invention has, in accordance with the invention, a greater specific surface area than the old deinstalled catalyst. In general, the activity thereof is also increased compared to the deinstalled catalyst.

The invention further provides a styrene catalyst obtainable by one of the above-described processes. The new catalyst obtained from recycled deinstalled catalyst is used in the dehydrogenation of ethylbenzene to styrene with water vapor in exactly the same way as a catalyst produced only from new raw materials.

### EXAMPLES

### Example 1

The deinstalled catalyst from a styrene reactor, after a run time of about 3 years, had a composition (in % by weight as metals) Fe: 48.0; K: 8.0; Ce: 7.2; Mg: 1.1; Ca: 1.5; residual content of other promoters, oxygen and carbon. The specific surface area (measured to DIN 66131/1973 by the 5-point BET method) was 2.7 m²/g. The phase composition of the deinstalled catalyst, determined by XRD analysis (Cu K-alpha cathode), exhibits the following crystallographic phases: magnetite Fe₃O₄, cerianite CeO₂, potassium molybdate K₂MoO₄, potassium carbonate hydrate K₂CO₃×1.5 H₂O, kalicinite KHCO₃.

675 g of this ground deinstalled styrene catalyst were stirred with 3820 g of 37% hydrochloric acid (pH=−1.1) under reflux at 60° C. for 24 h. This dissolved all of the catalyst except small residues of carbon, which were removed by filtration. Thereafter, the solution was precipitated with 3540 g of 50% sodium hydroxide solution (NaOH). The solids formed were filtered off and washed with 10 l of demineralized water. This gave 514 g of solids containing 8.2% by weight of Ce, 0.009% by weight of K, 0.1% by weight of Mg and 0.06% by weight of Ca. 470 g of these solids were mixed with 119 g of potassium carbonate hydrate, 10 g of magnesite and 13 g of calcium hydroxide, kneaded with starch solution, extruded and calcined at 825° C. This gave a catalyst with a specific surface area of 5.3 m²/g.

### Example 2

675 g of deinstalled styrene catalyst (composition as described in example 1) were stirred with 3820 g of 37% hydrochloric acid (pH=−1.1) under reflux at 60° C. for 24 h. This dissolved all of the catalyst except small residues of carbon, which were removed by filtration. The solution was dried at 340° C. in a spray tower. The resulting solids comprised 5.9% by weight of Ce, 4.8% by weight of K, 0.91% by weight of Mg, 1.20% by weight of Ca and 25.8% by weight of Cl. To reduce the Cl content, the solids were calcined at 870° C. for 1 h. The solids subsequently still comprised 0.12% by weight of Cl and 8.0% by weight of Ce, 3.7% by weight of K, 1.3% by weight of Mg, 1.4% by weight of Mo, 1.5% by weight of Ca, and had a specific surface area of 1.2 m²/g.

### Example 3

10 g of ground deinstalled catalyst (composition as described in example 1) were suspended with 20 g of ascorbic acid and 20 g of oxalic acid in 250 ml of demineralized water and stirred at 25° C. and a pH of 0.5 for 12 h. The resulting white suspension of organic salts was centrifuged, dried and calcined at 400° C. The resulting solids comprised 7.3% by weight of Ce, 7.5% by weight of K, 1.2% by weight of Mg, 1.3% by weight of Mo, 1.6% by weight of Ca, and had a specific surface area of 11 m²/g.

### Example 4

10 g of ground deinstalled catalyst (composition as described in example 1) were stirred under reflux with 4 g of oxalic acid and 25 ml of demineralized water at 60° C. and a pH of 0.2 for 24 h. At the end, a portion of the magnetite remained undissolved. The suspension was dried and calcined at 825° C. The resulting solids comprised 7.2% by weight of Ce, 8.3% by weight of K, 1.1% by weight of Mg, 1.4% by weight of Mo, 1.5% by weight of Ca, and had a specific surface area of 4.8 m²/g.

1-9. (canceled)

10. A process for producing a new styrene catalyst from a spent styrene catalyst, comprising the steps of
(i) treating the spent styrene catalyst with an aqueous acid and optionally a reducing agent to obtain an aqueous solution comprising the soluble constituents of the catalyst or a suspension,
(ii) converting the aqueous solution or suspension to a solid,
(iii) adding any missing constituents after analysis,
(iv) shaping and calcining the solid mixture obtained in step (iii).

11. The process according to claim 10, wherein the spent cerium-containing catalyst comprises at least compounds of the metals iron, corresponding to 40 to 90% by weight of Fe₂O₃, potassium, corresponding to 1 to 40% by weight of K₂O and cerium, corresponding to 1 to 25% by weight of CeO₂.

12. The process according to claim 11, wherein the spent cerium-containing catalyst additionally comprises compounds of calcium, corresponding to 0 to 10% by weight of CaO, molybdenum, corresponding to 0.1 to 10% by weight of MoO₃, and/or magnesium, corresponding to 0 to 10% by weight of MgO.

13. The process according to claim 10, wherein the spent styrene catalyst prior to the treatment with an aqueous acid is mechanically comminuted, washed with water and/or calcined in an oxygenous atmosphere.

14. The process according to claim 10, wherein an aqueous acid with a pH of <0.5 is used.

15. The process according to claim 10, wherein the new styrene catalyst is shaped to rings, cylinders, stars or honeycombs.

16. The process according to claim 10, wherein the new styrene catalyst has a greater specific surface area than the spent styrene catalyst.

17. A styrene catalyst obtainable by the process according to claim 10.

18. The use of the new styrene catalyst obtainable by the process according to claim 10 for dehydrogenation of ethylbenzene.
